(19) United States
(12) Patent Application Publication
(10) Pub. No.: US 2023/0009259 A1
(43) Pub. Date: Jan. 12, 2023

(54) Methods of Treating Eye Disorders

(71) Applicant: Baradaina LLC, Niles, IL (US)

(72) Inventor: John C. MICHAEL, Niles, IL (US)

(73) Assignee: Baradaina LLC, Niles, IL (US)

(21) Appl. No.: 17/934,942

(22) Filed: Sep. 23, 2022

Related U.S. Application Data
(63) Continuation of application No. 16/825,115, filed on Mar. 20, 2020.
(60) Provisional application No. 62/822,362, filed on Mar. 22, 2019.

(51) Int. Cl. A61K 31/496; A61K 9/00

(52) U.S. Cl. CPC A61K 31/496; A61K 9/0019; A61K 9/0048

(57) ABSTRACT

The present invention is related to a method of treating a patient having an eye disorder selected from the group consisting of presbyopia, glaucoma, or halos and glare, the method comprising administering to the patient a liquid composition comprising a pharmacologically effective amount of dapiprazole or its pharmaceutically acceptable salt.

### RELATED APPLICATION

This application claims benefit to U.S. Provisional Application No. 62/822,362, filed on Mar. 22, 2019, which is hereby incorporated by reference.

### BACKGROUND

Presbyopia is the normal loss of near focusing ability that occurs with age. It is generally believed to stem from a gradual thickening and loss of flexibility of the natural lens inside the eyes. Eyeglasses with progressive lenses or bifocal lenses are the most popular solution for presbyopia for most people over age 40. A number of surgical options to treat presbyopia are available as well. For example, implantation of a corneal inlay can increase depth of focus of the treated eye and reduces the need for reading glasses without significantly affecting the quality of the patient's distance vision.

Glaucoma is a group of eye conditions that is the result of damage to the optic nerve, which is normally caused by an abnormally high pressure in the eye. Glaucoma is one of the leading causes of blindness for people over the age of 60. It can occur at any age but is more common in older adults. Closed-angle glaucoma occurs when the iris bulges forward to narrow or block the drainage angle formed by the cornea and iris. As a result, fluid can't circulate through the eye and pressure increases. In pigmentary glaucoma, pigment granules from the iris build up in the drainage channels, slowing or blocking fluid exiting the eye. Glaucoma treatments include medicines, laser trabeculoplasty, conventional surgery, or a combination of any of these.

Halos and glare are the two most common vision defects people experience. Halos appear as bright circles around sources of light. Glare occurs in a vision when light enters the eye and actually obstructs the vision. Halos and glare are common side effects following LASIK surgery. Patients undergoing LASIK procedures display an increase of halos and glare phenomena around lights in night vision conditions, even when the results of the surgery are considered entirely satisfactory according to current standards. Common treatments for halos and glare include sunglasses and lenses.

Current therapy for the above eye disorders often requires the patient to wear burdensome equipment or involves painful surgeries. There is a need for an effective and convenient method for treating these eye disorders.

### SUMMARY OF THE INVENTION

Provided herein are methods of treating a patient having an eye disorder selected from the group consisting of presbyopia, glaucoma, or halos and glare, wherein the method comprises administering to the patient a liquid composition comprising a pharmacologically effective amount of dapiprazole or its pharmaceutically acceptable salt.

In some embodiments, the eye disorder is presbyopia.

In some embodiments, the eye disorder is glaucoma. In some embodiments, the glaucoma is pigmentary glaucoma. In still some embodiments, the glaucoma is closed angle glaucoma. In still some embodiments, the eye disorder is pigment dispersion syndrome.

In some embodiments, the eye disorder is halos and glare. In some embodiments, the halos and glare is caused by LASIK photorefractive surgery.

In some embodiments, the liquid composition is administered to the patient by intraretinal injection. In some embodiments, the liquid composition is administered to the patient by intravitreal injection.

In some embodiments, the liquid composition is administered in an eye drop formulation.

In some embodiments, the pharmaceutically acceptable salt of dapiprazole is dapiprazole hydrochloride.

In some embodiments, the liquid composition is administered at a dosage of about 0.1 μg/kg/day to about 3 mg/kg/day of dapiprazole or its pharmaceutically acceptable salt. In some embodiments, the liquid composition is administered at a dosage of about 0.1 μg/kg/day to about 0.5 mg/kg/day of dapiprazole or its pharmaceutically acceptable salt. In some embodiments, the liquid composition is administered at a dosage of about 0.1 μg/kg/day to about 5 μg/kg/day of dapiprazole or its pharmaceutically acceptable salt.

In some embodiments, the liquid composition comprises about 0.1% to about 5% by weight of dapiprazole or its pharmaceutically acceptable salt. In some embodiments, the liquid composition comprises about 0.1% to about 2% by weight of dapiprazole or its pharmaceutically acceptable salt. In some embodiments, the liquid composition comprises about 0.1% to about 1% by weight of dapiprazole or its pharmaceutically acceptable salt.

In some embodiments, about 50 μl to about 0.5 ml of the liquid composition is administered to the patient. In some embodiments, about 50 μl to about 0.2 ml of the liquid composition is administered to the patient.

In some embodiments, the liquid composition is administered using multiple drops.

In some embodiments, the liquid composition is administered to the patient as a one-time treatment. In some embodiments, the liquid composition is administered to the patient repetitively within a certain period of time.

In some embodiments, the liquid composition further comprises additional pharmaceutically acceptable excipients. In some embodiments, the liquid composition further comprises water.

### DETAILED DESCRIPTION OF THE INVENTION

Unless defined otherwise, all technical and scientific terms used herein have the same meaning as commonly understood by one of ordinary skill in the art to which this disclosure belongs. In case of conflict, the present application including the definitions will control.

In order to further define this disclosure, the following terms and definitions are provided.

The singular forms “a,” “an” and “the” include plural referents unless the context clearly dictates otherwise. In certain aspects, the term “a” or “an” means “single.” In other aspects, the term “a” or “an” includes “two or more” or “multiple.”

The term “pharmaceutically acceptable salt” refers to a form of dapiprazole that consists of a cationic form of dapiprazole in combination with a suitable anion. In some embodiments, the pharmaceutically acceptable salt of dapiprazole is a cationic form of dapiprazole in combination with a suitable anion. Examples of suitable anions include but are not limited to those derived from nitric acid, nitrous acid, phosphoric acid, sulfuric acid, sulphurous acid, hydrobromic acid, hydroiodic acid, hydrofluoric acid, phosphoric acid, and phosphorous acids.

The term “treating” as used herein refers to the administration of a composition to a subject for therapeutic purposes.

The term “administration” or “administering” as used herein refers to the act of a physician or other medical professional prescribing a pharmaceutical composition of the invention for a subject.

One embodiment of this invention is a method of treating a patient having an eye disorder selected from the group consisting of presbyopia, glaucoma, or halos and glare, wherein the method comprises administering to the patient a liquid composition comprising a pharmacologically effective amount of dapiprazole or its pharmaceutically acceptable salt.

In some embodiments, the eye disorder is presbyopia. In some embodiments, the eye disorder is glaucoma. In some embodiments, the glaucoma is pigmentary glaucoma. In still some certain embodiments, the glaucoma is closed angle glaucoma. In certain embodiments, the eye disorder is pigment dispersion syndrome. In some embodiments, the eye disorder is halos and glare. In some embodiments, the halos and glare is caused by LASIK photorefractive surgery. In some embodiments, the eye disorder is dry eyes, double vision, astigmatism, open angle glaucoma, normal tension glaucoma, secondary glaucoma, congenital glaucoma, pseudoexfoliative glaucoma, traumatic glaucoma, neovascular glaucoma, irido Corneal endothelial syndrome, or uveitic glaucoma.

In some embodiments, the liquid composition is administered to the patient by intraretinal injection. In some embodiments, the liquid composition is administered to the patient by intravitreal injection. In some embodiments, the liquid composition is administered in an eye drop formulation.

In some embodiments, the pharmaceutically acceptable salt of dapiprazole is dapiprazole hydrochloride.

In some embodiments, the liquid composition is administered at a dosage of about 0.1 μg/kg/day to about 3 mg/kg/day of dapiprazole or its pharmaceutically acceptable salt. In some embodiments, the liquid composition is administered at a dosage of about 0.1 μg/kg/day to about 0.5 mg/kg/day of dapiprazole or its pharmaceutically acceptable salt. In some embodiments, the liquid composition is administered at a dosage of about 0.1 μg/kg/day to about 5 μg/kg/day of dapiprazole or its pharmaceutically acceptable salt. In some embodiments, the liquid composition is administered at a dosage of about 0.05 μg/kg/day to about 5 mg/kg/day, about 0.05 μg/kg/day to about 4 mg/kg/day, about 0.05 μg/kg/day to about 3 mg/kg/day, about 0.05 μg/kg/day to about 2 mg/kg/day, about 0.05 μg/kg/day to about 1 mg/kg/day, about 0.05 μg/kg/day to about 0.5 mg/kg/day, about 0.05 μg/kg/day to about 0.2 mg/kg/day, about 0.05 μg/kg/day to about 100 μg/kg/day, about 0.05 μg/kg/day to about 20 μg/kg/day, about 0.05 μg/kg/day to about 5 μg/kg/day, about 0.1 μg/kg/day to about 5 mg/kg/day, about 0.1 μg/kg/day to about 4 mg/kg/day, about 0.1 μg/kg/day to about 2 mg/kg/day, about 0.1 μg/kg/day to about 1 mg/kg/day, about 0.1 μg/kg/day to about 0.2 mg/kg/day, about 0.1 μg/kg/day to about 100 μg/kg/day, about 0.1 μg/kg/day to about 20 μg/kg/day, about 0.5 μg/kg/day to about 5 mg/kg/day, about 0.5 μg/kg/day to about 4 mg/kg/day, about 0.5 μg/kg/day to about 3 mg/kg/day, about 0.5 μg/kg/day to about 2 mg/kg/day, about 0.5 μg/kg/day to about 1 mg/kg/day, about 0.5 μg/kg/day to about 0.5 mg/kg/day, about 0.5 μg/kg/day to about 0.2 mg/kg/day, about 0.5 μg/kg/day to about 100 μg/kg/day, about 0.5 μg/kg/day to about 20 μg/kg/day, about 0.5 μg/kg/day to about 5 μg/kg/day, about 2 μg/kg/day to about 5 mg/kg/day, about 2 μg/kg/day to about 4 mg/kg/day, about 2 μg/kg/day to about 3 mg/kg/day, about 2 μg/kg/day to about 2 mg/kg/day, about 2 μg/kg/day to about 1 mg/kg/day, about 2 μg/kg/day to about 0.5 mg/kg/day, about 2 μg/kg/day to about 0.2 mg/kg/day, about 2 μg/kg/day to about 100 μg/kg/day, about 2 μg/kg/day to about 20 μg/kg/day, about 2 μg/kg/day to about 5 μg/kg/day, about 5 μg/kg/day to about 5 mg/kg/day, about 5 μg/kg/day to about 4 mg/kg/day, about 5 μg/kg/day to about 3 mg/kg/day, about 5 μg/kg/day to about 2 mg/kg/day, about 5 μg/kg/day to about 1 mg/kg/day, about 5 μg/kg/day to about 0.5 mg/kg/day, about 5 μg/kg/day to about 0.2 mg/kg/day, about 5 μg/kg/day to about 100 μg/kg/day, about 5 μg/kg/day to about 20 μg/kg/day, about 50 μg/kg/day to about 5 mg/kg/day, about 50 μg/kg/day to about 4 mg/kg/day, about 50 μg/kg/day to about 3 mg/kg/day, about 50 μg/kg/day to about 2 mg/kg/day, about 50 μg/kg/day to about 1 mg/kg/day, about 50 μg/kg/day to about 0.5 mg/kg/day, about 50 μg/kg/day to about 0.2 mg/kg/day, about 50 μg/kg/day to about 100 μg/kg/day, about 0.5 mg/kg/day to about 5 mg/kg/day, about 0.5 mg/kg/day to about 4 mg/kg/day, about 0.5 mg/kg/day to about 3 mg/kg/day, about 0.5 mg/kg/day to about 2 mg/kg/day, about 0.5 mg/kg/day to about 1 mg/kg/day, about 1 mg/kg/day to about 5 mg/kg/day, about 1 mg/kg/day to about 4 mg/kg/day, about 1 mg/kg/day to about 3 mg/kg/day, or about 1 mg/kg/day to about 2 mg/kg/day of dapiprazole or its pharmaceutically acceptable salt.

In some embodiments, the liquid composition comprises about 0.1% to about 5% by weight of dapiprazole or its pharmaceutically acceptable salt. In some embodiments, the liquid composition comprises about 0.1% to about 2% by weight of dapiprazole or its pharmaceutically acceptable salt. In some embodiments, the liquid composition comprises about 0.1% to about 1% by weight of dapiprazole or its pharmaceutically acceptable salt. In some embodiments, the liquid composition comprises about 0.05% to about 5%, about 0.05% to about 4%, about 0.05% to about 3%, about 0.05% to about 2%, about 0.05% to about 1%, about 0.05% to about 0.5%, about 0.05% to about 0.2%, about 0.1% to about 4%, about 0.1% to about 3%, about 0.1% to about 0.5%, about 0.5% to about 5%, about 0.5% to about 4%, about 0.5% to about 3%, about 0.5% to about 2%, about 0.5% to about 1%, about 1% to about 5%, about 1% to about 4%, about 1% to about 3%, about 1% to about 2%, about 2% to about 5%, about 2% to about 4%, or about 2% to about 3% by weight of dapiprazole or its pharmaceutically acceptable salt.

In some embodiments, about 50 μl to about 0.5 ml of the liquid composition is administered to the patient. In some embodiments, about 50 μl to about 0.2 ml of the liquid composition is administered to the patient. In some embodiments, about 20 μl to about 0.5 ml, about 20 μl to about 0.4 ml, about 20 μl to about 0.3 ml, about 20 μl to about 0.2 ml, about 20 μl to about 0.1 ml, about 20 μl to about 50 about 50 μl to about 0.4 ml, about 50 μl to about 0.3 ml, about 50 μl to about 0.1 ml, about 75 μl to about 0.5 ml, about 75 μl to about 0.4 ml, about 75 μl to about 0.3 ml, about 75 μl to about 0.2 ml, about 75 μl to about 0.1 ml, about 0.1 ml to about 0.5 ml, about 0.1 ml to about 0.4 ml, about 0.1 μl to about 0.3 ml, about 0.1 ml to about 0.2 ml, about 0.2 ml to about 0.5 ml, about 0.2 ml to about 0.4 ml, about 0.2 ml to about 0.3 ml, about 0.3 μl to about 0.5 ml, about 0.3 ml to about 0.4 ml, or about 0.4 ml to about 0.5 ml of the liquid composition is administered to the patient.

In some embodiments, the liquid composition is administered using one or multiple drops. The multiple drops can be administered on the same day or separated by one or more hours, days, weeks, or months. In some embodiments, the liquid composition is administered using less than 25 drops, less than 20 drops, less than 15 drops, less than 10 drops, less than 5 drops, or less than 3 drops.

In some embodiments, the liquid composition is administered to the patient as a one-time treatment. In some embodiments, the liquid composition is administered to the patient repetitively within a certain period of time. In some embodiments, the liquid composition is administered daily for two or more days. In some embodiments, the liquid composition is administered two or more times a day within a certain period of time. In some embodiments, the liquid composition is administered two or more times a day within a certain period of time for two or more days. In some embodiments, the liquid composition is administered to the patient as needed. In some embodiments, the liquid composition is administered to the patient once, twice, or three times daily. In some embodiments, the liquid composition is administered to the patient more than three times daily.

In some embodiments, the liquid composition further comprises additional pharmaceutically acceptable excipients. In some embodiments, the liquid composition contains one more water soluble extenders. Examples of suitable extenders include but are not limited to mannitol, sucrose, lactose, and glycine. In some embodiments, the liquid composition includes a tonicity agent. Examples of suitable tonicity agents include but are not limited to sodium chloride and potassium chloride. In some embodiments, the liquid composition contains polymers. Examples of suitable polymers include but are not limited to hydroxypropyl methylcellulose, hydroxyethyl cellulose, hydroxypropyl cellulose, and sodium carboxymethylcellulose. In some embodiments, the liquid composition contains a buffer agent. Examples of suitable buffer agents include but are not limited to edetate sodium, sodium phosphate dibasic, sodium phosphate monobasic, and sodium citrate dihydrate. In some embodiments, the liquid composition contains a preservative. Examples of suitable preservatives include but are not limited to benzalkonium chloride and polyquad. In some embodiments, the liquid composition further comprises water.

In some embodiments, the composition has a pH of between about 5.0 and about 8.0. In some embodiments, the composition has a pH of between about 6.0 and about 8.0. In some embodiments, the composition has a pH of about 7.4. In some embodiments, the composition has a pH of about 6.6. In some embodiments, the composition comprises a buffer for maintaining the pH. Examples of suitable buffers include, but are not limited to, phosphate buffers, citrate buffers, borate buffers, acetate buffers, and mixtures thereof.

### EXAMPLES

### Example 1

In accordance with the present disclosure, the following composition is prepared: dapiprazole hydrochloride (0.5%), mannitol, sodium chloride, hydroxypropyl methylcellulose (0.4%), edetate sodium (0.01%), sodium phosphate dibasic, sodium phosphate monobasic, water for injection, and benzalkonium chloride (0.01%) as a preservative.

The breadth and scope of the present invention should not be limited by any of the above-described exemplary embodiments, but should be defined only in accordance with the following claims and their equivalents.

1. A method of treating a patient having an eye disorder selected from the group consisting of presbyopia, glaucoma, or halos and glare, the method comprising administering to the patient a liquid composition comprising a pharmacologically effective amount of dapiprazole or its pharmaceutically acceptable salt.

2. The method of claim 1, wherein the eye disorder is presbyopia.

3. The method of claim 1, wherein the eye disorder is glaucoma.

4. The method of claim 3, wherein the glaucoma is pigmentary glaucoma.

5. The method of claim 3, wherein the glaucoma is closed angle glaucoma.

6. The method of claim 1, wherein the eye disorder is pigment dispersion syndrome.

7. The method of claim 1, wherein the eye disorder is halos and glare.

8. The method of claim 7, wherein the halos and glare is caused by LASIK photorefractive surgery.

9. The method of claim 1, wherein the liquid composition is administered to the patient by intraretinal injection.

10. The method of claim 1, wherein the liquid composition is administered to the patient by intravitreal injection.

11. The method of claim 1, wherein the liquid composition is administered in an eye drop formulation.

12. The method of claim 1, wherein the pharmaceutically acceptable salt of dapiprazole is dapiprazole hydrochloride.

13. The method of claim 1, wherein the liquid composition is administered at a dosage of about 0.1 μg/kg/day to about 3 mg/kg/day of dapiprazole or its pharmaceutically acceptable salt.

14. The method of claim 1, wherein the liquid composition is administered at a dosage of about 0.1 μg/kg/day to about 0.5 mg/kg/day of dapiprazole or its pharmaceutically acceptable salt.

15. The method of claim 1, wherein the liquid composition is administered at a dosage of about 0.1 μg/kg/day to about 5 μg/kg/day of dapiprazole or its pharmaceutically acceptable salt.

16. The method of claim 1, wherein the liquid composition comprises about 0.1% to about 5% by weight of dapiprazole or its pharmaceutically acceptable salt.

17. The method of claim 1, wherein the liquid composition comprises about 0.1% to about 2% by weight of dapiprazole or its pharmaceutically acceptable salt.

18. The method of claim 1, wherein the liquid composition comprises about 0.1% to about 1% by weight of dapiprazole or its pharmaceutically acceptable salt.

19. The method of claim 1, wherein about 50 μl to about 0.5 ml of the liquid composition is administered to the patient.

20. The method of claim 1, wherein about 50 μl to about 0.2 ml of the liquid composition is administered to the patient.

21. The method of claim 19, wherein the liquid composition is administered using multiple drops.

22. The method of claim 1, wherein the liquid composition is administered to the patient as a one-time treatment.

23. The method of claim 1, wherein the liquid composition is administered to the patient repetitively within a certain period of time.

24. The method of claim 1, wherein the liquid composition further comprises additional pharmaceutically acceptable excipients.

25. The method of claim 1, wherein the liquid composition further comprises water.
